(19) United States
(12) Patent Application Publication
(10) Pub. No.: US 2026/0053494 A1
(43) Pub. Date: Feb. 26, 2026

(54) SYSTEMS AND METHODS FOR ENDOSCOPIC TISSUE GRASPING

(71) Applicant: INTUITIVE SURGICAL OPERATIONS, INC., Sunnyvale, CA (US)

(72) Inventors: Thad W. Lieb, Santa Barbara, CA (US); Thomas W. Brown, Santa Clara, CA (US); Jennifer A. Hayden, San Jose, CA (US); Kyle R. Miller, San Jose, CA (US); Tabish Mustufa, Sunnyvale, CA (US); Bruce M. Schena, Menlo Park, CA (US)

(21) Appl. No.: 19/103,748

(22) PCT Filed: Aug. 15, 2023
(86) PCT No.: PCT/US2023/030202

§ 371 Date: Feb. 13, 2025

Related U.S. Application Data
(60) Provisional application No. 63/398,454, filed on Aug. 16, 2022.

(51) Int. Cl. A61B 17/04; A61B 17/00; A61B 17/06; A61B 90/00; A61F 5/00

(52) U.S. Cl. CPC A61B 17/0469; A61B 17/0482; A61B 17/06066; A61F 5/0076; A61F 5/0089; A61B 2017/00022; A61B 2017/00057; A61B 2017/0472; A61B 2017/06076; A61B 2090/064

(57) ABSTRACT

A system may comprise an elongate flexible instrument, a tissue grasping device including a helical needle extending from a distal end of the elongate flexible instrument, and a grasper drive system coupled to the tissue grasping device to drive motion of the tissue grasping device. The system may also include a sensor system coupled to the tissue grasping device and a control system configured to receive sensor information from the sensor system and control the grasper drive system in response to the received sensor information.

### CROSS-REFERENCED APPLICATIONS

This application claims priority to and benefit of U.S. Provisional Application No. 63/398,454, filed Aug. 16, 2022 and entitled “Systems and Methods for Endoscopic Tissue Grasping,” which is incorporated by reference herein in its entirety.

### FIELD

Examples described herein relate to systems and methods for endoscopic tissue grasping. More particularly, examples may relate to tissue grasping and sensing during an endoscopic suturing procedure.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. Some minimally invasive medical instruments may be used to perform endoscopic suturing. Systems and methods are needed to provide effective suture purchase and retention.

### SUMMARY

The following presents a simplified summary of various examples described herein and is not intended to identify key or critical elements or to delineate the scope of the claims.

In some examples, a system may comprise an elongate flexible instrument, a tissue grasping device including a helical needle extending from a distal end of the elongate flexible instrument, and a grasper drive system coupled to the tissue grasping device to drive motion of the tissue grasping device. The system may also include a sensor system coupled to the tissue grasping device and a control system configured to receive sensor information from the sensor system and control the grasper drive system in response to the received sensor information.

In some examples, a method for operating a medical system may comprise inserting an elongate flexible instrument in an endoluminal passage and extending a tissue grasping device including a helical needle from the elongate flexible instrument into tissue. The method may also include receiving, from a sensor system, sensor information about penetration of the tissue grasping device into the tissue and controlling a drive system coupled to the tissue grasping device based on the received sensor information.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a patient anatomy, according to some examples.

FIG. 2 illustrates a distal portion of an endoscopic instrument system, according to some examples.

FIG. 3 illustrates a distal portion of an endoscopic instrument system, according to some examples.

FIGS. 4A-4F illustrate tissue grasping device configurations, according to some examples.

FIGS. 5A and 5B illustrate a tissue grasping device with a pressure sensor, according to some examples.

FIGS. 6A and 6B illustrate tissue grasping device with an optical sensor, according to some examples.

FIG. 7A is a cross-sectional view of tissue grasping device with an electrical sensor, according to some examples.

FIG. 7B is a side view of a tissue grasping device with an electrical sensor, according to some examples.

FIG. 7C is a layered view of the electrical sensor of FIG. 7B.

FIGS. 8A-8C illustrate a tissue grasping device with an optical fiber sensor, according to some examples.

FIG. 9 is a flowchart illustrating of a method of operating a medical system, according to some examples.

FIGS. 10A-10C illustrate tissue when a tissue grasping device is at different stages of insertion.

FIG. 11 is a robotically-assisted medical system, according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The technology described herein provides techniques and treatment systems for endoscopic tissue grasping and sensing. Although the examples provided herein may refer to suturing of stomach tissue or techniques for endoscopic sleeve gastroplasty (“ESG”), it is understood that the described technology may be used in performing procedures in artificially created lumens or any endoluminal passageway or cavity, including in a patient trachea, colon, intestines, stomach, liver, kidneys and kidney calices, brain, heart, circulatory system including vasculature, fistulas, and/or the like.

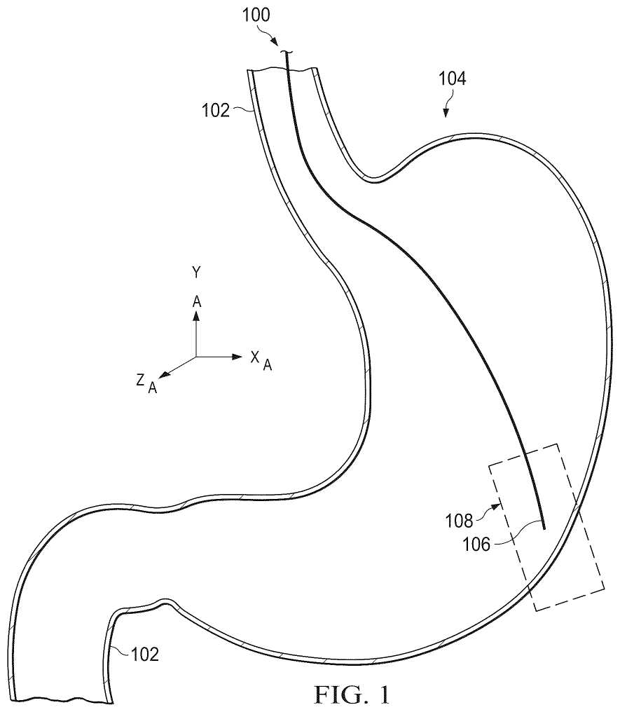

FIG. 1 illustrates an endoscopic instrument system 100 extending within anatomic passageways 102 of an anatomical structure 104. In some examples the anatomic structure 104 may be a stomach. The anatomic structure 104 has an anatomical frame of reference (X_{A}, Y_{A}, Z_{A}). A distal end portion 106 of the endoscopic instrument system 100 may be advanced into an anatomic opening (e.g., a patient mouth) and through the anatomic passageways 102 to perform a medical procedure, such as an endoscopic sleeve gastroplasty, at or near target tissue located in a region 108 of the anatomic structure 104.

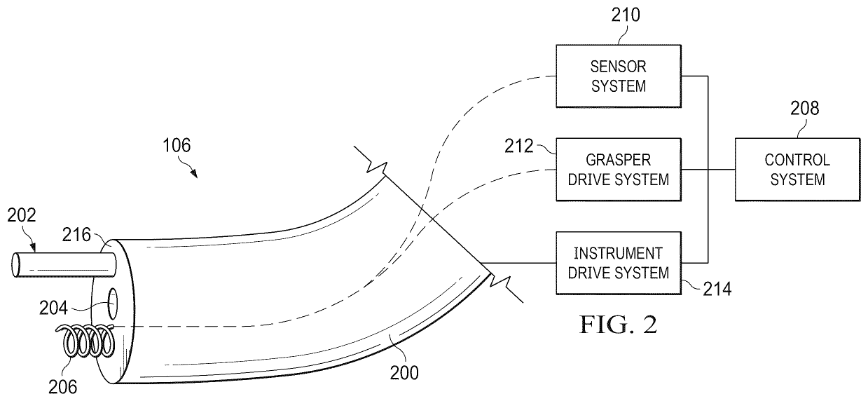

FIG. 2 illustrates the distal end portion 106 of the endoscopic instrument system 100. The endoscopic instrument system 100 may include an elongate flexible instrument 200. The elongate flexible instrument 200 may serve as a platform to introduce working components into the anatomic structures accessed by the endoscopic instrument system 100. In some examples, the working components include an instrument system 202 and/or a tissue grasping device 206. A working channel 204 may extend through the elongate flexible instrument 200 to provide passage for removable instrument systems and allow instruments to be exchanged during a procedure. The working channel may also or alternatively allow fluid passage or otherwise provide access between proximal and distal portions of the elongate flexible instrument. In some examples, the working channel 204 extends throughout elongate flexible instrument 200 and defines an opening in a distal end portion 216. In other examples, the working channel 204 may define an opening in a sidewall of elongate flexible instrument 200.

The instrument system 202 may be coupled to the distal end portion 216 of the elongate flexible instrument 200 or delivered through the elongate flexible instrument 200. As shown in the example of FIG. 2, the instrument system 202 may extend distally past a distal end 216 of the elongate flexible instrument 200. The instrument system 202 may include, for example, one or more of a suturing system, a biopsy system, a treatment system, or an ablation system.

The tissue grasping device 206 may be extendable distally from the distal end 216 of the elongate flexible instrument 200. In some examples, the tissue grasping device 206 is a helical needle grasping device that may be rotatably driven by a tissue grasping drive system 212 into tissue (e.g., a corkscrew type advancement into the tissue). The tissue grasping device 206 may grasp the tissue while the instrument system 202 performs a procedure such as suturing, biopsy, treatments, or ablation. In some examples, the instrument system 202 is a suturing instrument system and the tissue grasping device 206 is a helical needle grasping device that may be rotatably driven by the tissue grasping drive system 212 into tissue to hold the tissue during the suturing. In other examples, where the instrument system 202 includes a biopsy system, the grasping device 206 may be used to grasp tissue during the biopsy. In examples where the instrument system 202 includes a treatment system, grasping device 206 may be used to grasp tissue while visualizing and/or treating the grasped tissue. In examples where the instrument system 202 includes an ablation system, the grasping device 206 may be used to grasp tissue during ablation.

In some examples, the elongate flexible instrument 200 may include a plurality of instruments systems, working channels, and/or tissue grasping devices. For example, the elongate flexible instrument 200 may include a plurality of instrument systems 202 including an imaging system, an illumination system, an irrigation system, and/or an instrument sensor system. The plurality of instrument systems may be removably (e.g., slidably) inserted into the elongate flexible instrument through one or more working channels. In some examples, the plurality of instrument systems may extend through dedicated instrument channels in the elongate flexible instrument. In some examples, instrument systems or portions of instrument systems may be fixed to the elongate flexible instrument.

The endoscopic instrument system 100 may further include a control system 208 in communication with a sensor system 210, a tissue grasping drive system 212, and/or an instrument drive system 214. The sensor system 210 and tissue grasping drive system 212 may be coupled to tissue grasping device 206. The instrument drive system 214 may be coupled to the elongate flexible instrument 200 to drive axial motion (e.g., advancement, retraction), bending, and/or roll motions of the elongate flexible instrument. The control system 208 may be a closed-loop control system in electrical communication with sensor system 210, tissue grasping drive system 212, and/or instrument drive system 214. The control system 208 may control the advancement, retraction, steering, and/or rotational motions of the endoscopic instrument system 100. In some examples, the control system 208 controls the elongate flexible instrument 200 and/or the instrument system 202 with instrument drive system 214 which may include motors, control cables, sensors, actuators, and/or other mechanical or electrical systems that drive motion in response to control signals from the control system 208. Further, the control system 208 may control the advancement, retraction, rotation or other motion of the tissue grasping device 206. In some examples, the control system 208 controls the tissue grasping device 206 with the tissue grasping drive system 212. The tissue grasping drive system 212 may include motors, control cables, sensors, actuators, and/or other mechanical or electrical systems that drive motion of the tissue grasping device 206 in response to control signals from the control system 208.

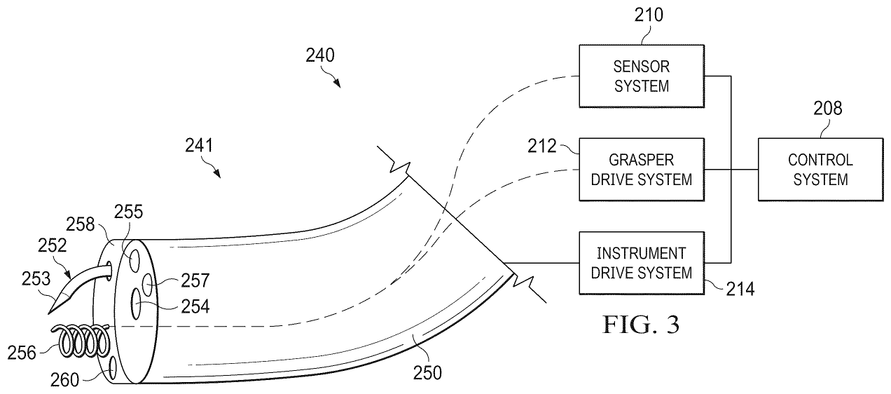

FIG. 3 illustrates a distal end portion 241 of an endoscopic instrument system 240 that may be used for suturing. The endoscopic instrument system 240 may be an example of an endoscopic instrument system 100 and may be substantially similar to system 100, with differences as described below. In this example, the endoscopic instrument system 240 may include an elongate flexible instrument 250 that includes a working channel 254 extending therethrough. An imaging system 255 and an illumination system 257 may also extend within the elongate flexible instrument 250. The endoscopic instrument system 240 may also include a suturing system housing 258 coupled to distal end portion of the elongate flexible instrument 250. The suturing system housing 258 carries a helical needle grasping device 256 (e.g. a grasping device 206) and an instrument system 252 (e.g., an instrument system 202) including a needle 253. The suturing system housing 258 may also include a capture port 260 into which the needle 253 may extend to secure the suture. The endoscopic instrument system 240 may include the control system 208, the sensor system 210, the grasper drive system 212, and the instrument drive system 214, as previously described.

In some examples, the endoscopic instrument system 240 may be used to perform an endoscopic sleeve gastroplasty (“ESG”) procedure. ESG is a weight loss/diabetes treatment procedure that uses an endoscopic suturing device to restrict/reduce the size of the stomach. The endoscopic instrument 240 may be inserted into mouth and navigated down to the interior of the stomach. The stomach wall is folded from the inside to effectively reduce the size of the stomach. By reducing the size of the stomach, the number of calories that can be absorbed by the body is reduced as well as the amount of food that is eaten. ESG is a non-surgical procedure that causes stomach reduction by folding the stomach without removing portions of the stomach. ESG also uses a natural body orifice for reaching the anatomy without an external incision.

In ESG procedures, the stomach tissue may be grasped from the interior of the stomach and retracted to create tissue folds. The tissue grasping device 256 may pull and retract the stomach wall. Once the tissue grasping device has sufficiently grasped the stomach wall and retracted/folded the stomach tissue, a separate needle may be used to penetrate and apply sutures to the folded tissue. One difficulty in ESG procedures is how to grasp the stomach wall from the inside with a purchase or bite of the tissue sufficient to cause tissue folding. The interior of the stomach may be slippery with a mucosal layer that is difficult to maintain in grasp, and the stomach wall may also include tough tissue that is resistant to folding. When using a tissue grasping device to create a fold, a preferred depth or range of depths for stomach wall penetration by the tissue grasping device may cause sufficient tissue folding. The stomach wall has at least four layers, the mucosal layer (innermost), submucosal layer, muscularis layer, and serosa (outermost layer). In some cases, penetration to the serosa may result in a full thickness bite through the entire stomach wall. In other cases, penetration may extend only to one of the inner layers, for example penetrating through the muscularis layer without penetrating through the serosa, to avoid penetrating tissue/organs outside of the stomach wall. A penetration or bite with the tissue grasping device that is too shallow may make it difficult to fold the tissue properly. A penetration or bite that is too deep may risk puncturing organs outside of the stomach.

The sensing system 210 and the control system 208 may allow the tissue grasping device 256 to achieve a firm grasp of the stomach wall without damaging tissue outside of the stomach. The sensor system 210 coupled to the helical needle grasping device 256 may detect when the helical needle has been driven through tissue to a target tissue depth. In some examples, when the sensor system 210 senses that the grasping device 256 is at the target tissue depth, the suturing needle 253 from the instrument system 252 may sweep through the grasped tissue to obtain a full-thickness suture bite.

Control system 208 may control a grasper drive system 212 to control the speed at which the grasping device 256 is inserted or retracted from the elongate flexible instrument 250. Further, the control system 208 may control the grasper drive system 212 to control the depth that grasping device 256 penetrates tissue. In other examples, the control system 208 provides an actuation signal to control the starting and/or stopping of the advancement and/or retraction of grasping device 256. For example, the control system 208 may be configured to advance the grasping device 256 when grasping device 256 is touching tissue. In another example, the control system 208 is configured to stop the grasping device 256 when the distal tip of the grasping device 256 is no longer touching tissue. The control system may reduce the speed of insertion of grasping device 256 when sensor system 210 senses the distal tip of grasping device 256 nearing the desired depth of insertion.

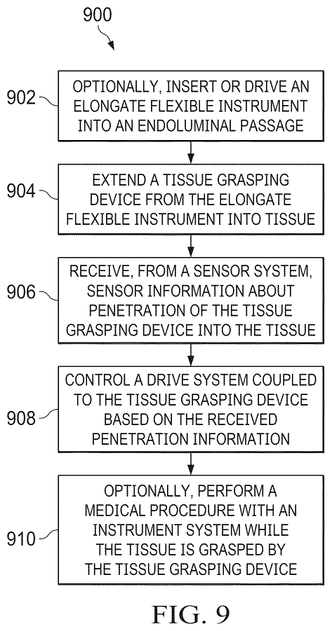

In some examples, the control system 208 may provide automated control over the insertion of grasping device 256 to reach a target grasping depth based on sensor data from the sensor system 210. The target grasping depth may be defined as a full thickness suture bite through an organ, a depth to a certain measurement (e.g., in mms), a depth to a certain number of rotations of grasping device 256, or depth to a particular tissue layer (e.g., specifying depth to reach within the muscularis, or depth for penetrating through the muscularis but not all the way through the serosa, etc.). A method for using the endoscopic instrument system 240 is described further below with respect to FIG. 9.

The control system 208 may control the grasper drive system 212 based on sensor information received from sensor system 210. In some examples, the received sensor information from sensor system 210 is displayed on a user interface (e.g., display system 1310). The user interface may be in communication with the control system 208 and provide feedback on the penetration of grasping device 256 (e.g., informing the user of positioning or instructing the user to adjust or try the bite again). In some examples, a user interface can provide an identification of the layer that grasping device 256 has entered. In other examples, the user interface can provide a side-view/cross-section representation correlated to the needle depth. For example, when activated, the user interface may include a cross-section that shows a representation of the approximate needle position relative to an estimated tissue thickness.

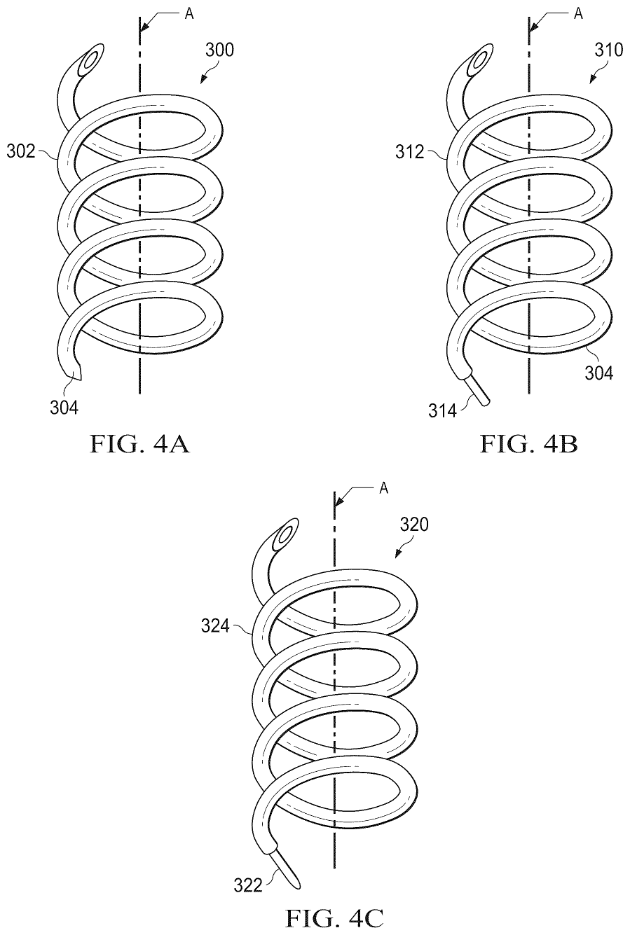

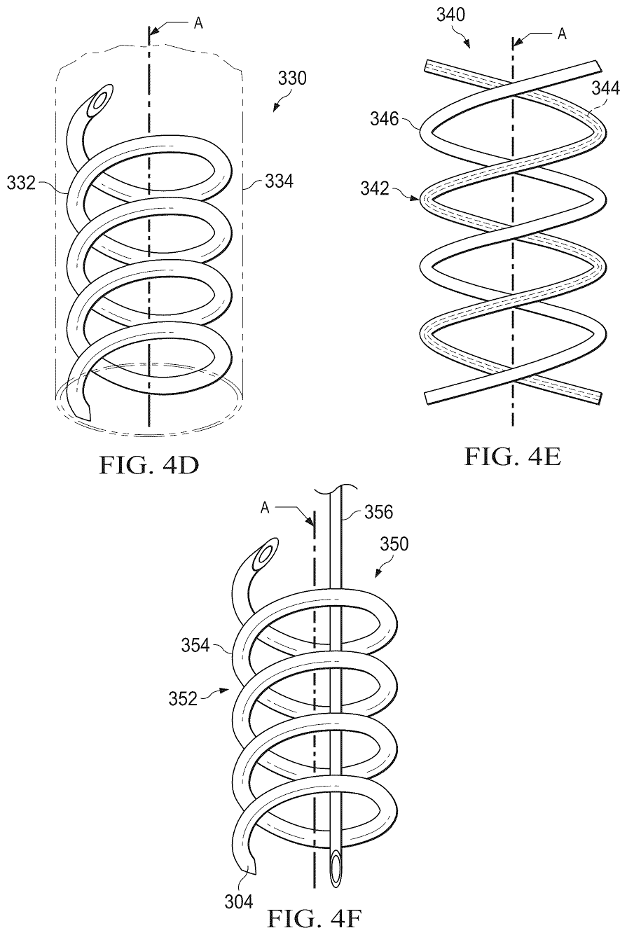

FIGS. 4A-4F illustrate configurations of a helical needle grasping device (e.g., grasping device 206, 256), according to some examples. FIGS. 4A-4F show grasping devices in the shape of a helix or corkscrew, but other grasping devices may achieve penetration and tissue grasp using other shapes. For example, a curved needle having one or more arcuate portions may be used and may be advantageous where insertion is not occurring generally perpendicular to an organ wall. Any of the features described for any of the grasping devices in FIGS. 4A-4F may be combined with other features of the grasping devices of FIGS. 4A-4F. FIGS. 4A-4F are only examples of possible configurations of a grasping device and are not meant to limit possible configurations. In some examples, the grasping device may be fabricated as a straight member and then shaped into a helix or other shape. In some examples, the grasping device may be a single use device not intended for sterilization or use on multiple patients. In other examples, the grasping device may be sterilized/reprocessed for use in multiple procedures.

FIG. 4A illustrates a grasping device 300 (e.g., grasping device 206, 256) that includes a single helically shaped needle 302 with a sharpened or angled distal tip 304. The helically shaped needle 302 may be rotated about an axis A to drive the needle into tissue. In some examples, the needle 302 may be cannulated to allow a sensor device, a fluid, or other material to reside within the needle for providing sensing information via the needle. A cannulated needle may have an opening at a distal end portion of the needle. In some examples, extending the grasping device 300 into tissue may include rotating the helical needle into the tissue while the needle advances longitudinally (along axis A) relative to the distal portion of the elongate flexible instrument 250. In some examples, extending the needle into tissue may include rotating the helical needle into the tissue while the needle remains longitudinally stationary relative to the distal portion of the elongate flexible instrument 250 and the tissue is pulled toward the elongate flexible instrument 250. In some examples, extending the needle into tissue may include rotating the helical needle into the tissue while the needle and tissue retract longitudinally (along the axis A) relative to the distal portion of the elongate flexible instrument 250.

FIG. 4B illustrates a grasping device 310 (e.g., grasping device 206, 256) that includes a cannulated helically shaped needle 312 through which a sensor device 314 extends or is positioned. The sensor device 314 may be a component of the sensor system 210. In some examples, the sensor device 314 includes a wire sensor or an optical fiber sensor that may be housed within the cannulated helical needle 312. In some examples, sensor device 314 may include an elongated plastic core member that is slidable with respect to the needle 312. The core 314 may be positioned to extend past a distal end of the distal tip 304 and may be retracted to be fully housed within the needle 312. The slidable core 314 may provide an indication of the amount of penetration by the needle 312 into tissue. For example, as the cannulated needle 312 is inserted into tissue, distal movement of slidable core 314 may be impeded by the tissue surface as the surrounding cannulated needle 312 is driven into the tissue. A distal end of the slidable core 314 may remain at the surface of the organ wall (e.g., the innermost surface of the stomach in an endoluminal application), while the needle 312 is inserted a distance into the tissue. The difference in advancement distance between the slidable core 314 and the needle 312 may provide a measure of the insertion distance of the needle 312 into the tissue. Markings may be placed on slidable core 314 or needle 312 to determine the differential measurement. The relative distance may be measured on either the proximal or distal portions of needle 312 or core 314. For example, a proximal end of needle 312 or a portion of the grasping device 310 proximal of the needle 312 may include a transparent or semi-transparent material, a window, or an opening, allowing the slidable core 314 extending therethrough and any markings on the slidable core 314 to be visualized. Alternatively, any portion of needle 312 may be transparent or semi-transparent to allow for viewing of markings on slidable core 314. In another example, the movement of the slidable core 314 relative to the needle 312 may be sensed with an electro-mechanical sensor of sensor system 210. The electro-mechanical sensor may be positioned, for example, on a proximal portion of needle 312 or within the grasper drive system 212.

FIG. 4C illustrates a grasping device 320 (e.g., grasping device 206, 256) that includes a helically shaped needle 322 over which a slidable sensor device 324 extends. The sensor device 324 may be a component of the sensor system 210. In some examples, the sensor device 324 is a sheath that extends over the helical needle 322 along the full or a partial length of the needle. The slidable sheath 324 may provide an indication of the distance travelled by the needle 322 into tissue. For example, as the needle 322 is inserted into tissue, the movement of slidable sheath 324 may be impeded by the tissue surface as the needle 322 is driven into the tissue. The slidable sheath 324 may remain at the surface of the organ wall (e.g., the innermost surface of the stomach in an endoluminal application), while the needle 322 is inserted a distance into tissue. The difference in advancement distance between the slidable sheath 324 and the needle 322 may provide a measure of the insertion distance of the needle 322 into the tissue. Markings may be placed on slidable sheath 324 or needle 322 to determine the differential measurement. For example, a distal end of slidable sheath 324 may include a transparent or semi-transparent material, a window, or an opening through which markings on the needle 322 can be visualized. In another example, the movement of the needle 322 relative to the slidable sheath 324 may be sensed with an electro-mechanical sensor of sensor system 210. In yet another example, markings on the proximal end of needle 322 may be viewed by an endoscope of the endoscopic instrument system 240.

FIG. 4D illustrates a grasping device 330 (e.g., grasping device 206, 256) that includes a helically shaped needle 332 around which a sheath 334 extends. In some examples, the sheath 334 may protect the needle 332 and surrounding tissue as the needle is extended distally of the elongate flexible instrument. The sheath 334 may be retractable along the axis A when the needle 332 is inserted into tissue. Alternatively, the sheath may remain at the surface of the tissue as the needle 332 is advanced distally into the tissue. In some examples, the sheath 334 may be a component of the sensor system 210. The sheath 324 may be slidable relative to the needle 332 to provide an indication of the distance travelled by the needle 332 into tissue. For example, as the needle 332 is inserted into tissue, the movement of slidable sheath 334 may be impeded by the tissue surface as the needle 332 is driven into the tissue. The slidable sheath 334 may remain at the surface of the organ wall (e.g., the innermost surface of the stomach in an endoluminal application), while the needle 332 is inserted a distance into tissue. The difference in advancement distance between the slidable sheath 334 and the needle 332 may provide a measure of the insertion distance of the needle 332 into the tissue. Markings may be placed on slidable sheath 334 or needle 332 to determine the differential measurement. The relative distance may be measured on either the proximal or distal portions of needle 332 or sheath 334.

FIG. 4E illustrates a grasping device 340 (e.g., grasping device 206, 256) that includes a double helix needle system 342 including needle 344 and needle 346. In some examples, one or both helical needles of the double helix needle system may be cannulated. In some examples, each needle 344, 346 may function with different sensing modalities. The helical needles 344, 346 may be wound in opposite directions about the axis A, as shown. In other examples, the helical needles may be wound in the same direction. In some examples, the helical needles may be nested (e.g., one needles coiled around the other) with one needle advanced and rotated before the other is advanced and rotated. In some examples, particularly if the helical needles are wound in the same direction, the diameter the helical needles may be different from each other to prevent the helical needles from interfering with each other. In some examples, the helical needles may have different pitches.

FIG. 4F illustrates a grasping device 350 (e.g., grasping device 206, 256) that includes a double needle system 352. The double needle system 352 may include helically shaped needle 354 wound around a straight or non-helically shaped needle 356 that may be aligned with or generally parallel to axis A. In some examples, the straight needle and the helical needle may each include components of the sensor system 210.

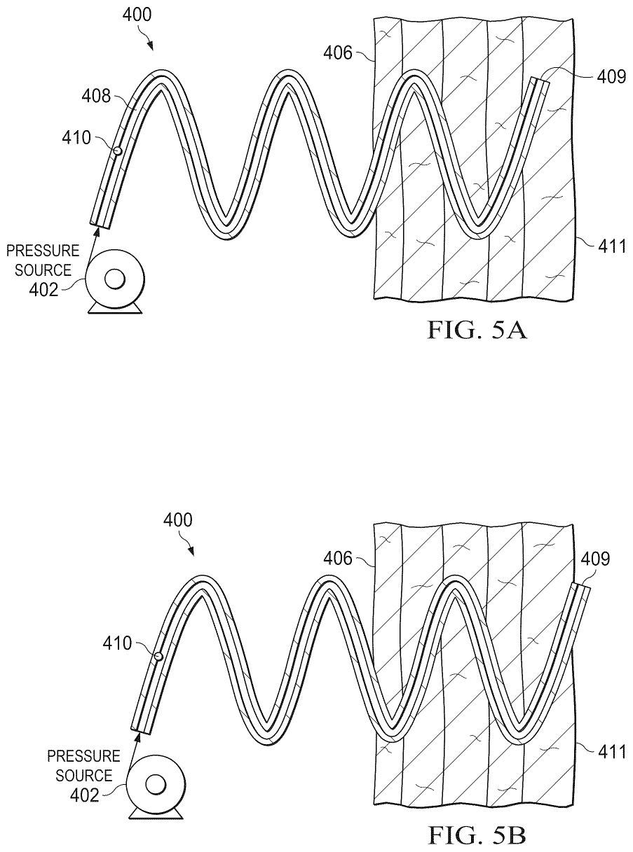

FIGS. 5A and 5B illustrate a grasping device 400 (e.g., grasping device 206, 256) used together with the sensing system 210 to determine penetration information about the grasping device into tissue 406. In some examples, the grasping device 400 is a helical, cannulated needle. In some examples, sensor system 210 may include a pressure sensor 410 and a pressure source 402. The pressure source 402 may include, for example, a pump that supplies a fluid such as air or another gas into the lumen of the cannulated grasping device 400. The pressure source 402 may include valves, regulators, and/or other control mechanisms to generate, monitor, and control fluid flow and pressure in the grasping device. The pressure source 402 and sensor 410 may be directly coupled to the needle 400, to another component of the endoscopic instrument system 100 or to a manual or robotically-assisted manipulator that controls operation of the endoscopic instrument system.

The pressure source 402 may provide air or another fluid into a lumen or channel 408 in the needle 400. The channel 408 may have a distal opening 409. As shown in FIG. 5A, as the needle 400 is advanced into tissue 406, the fluid will be prevented from exiting the channel 408 by tissue occluding the distal opening 409. Consequently, a back pressure in the needle 400, as measured by the pressure sensor 410, may rise. When the needle 400 penetrates through the outer surface 411 of the tissue 406 (e.g., the stomach wall), as shown in FIG. 5B, the distal opening 409 may no longer be occluded by the tissue 406. Consequently, the pressure in the needle 400, as measured by the pressure sensor 410, may drop. Thus, the pressure sensor 410 may provide penetration information indicating whether the distal tip of the needle 400 is within the tissue or has punctured through the outer surface 411 of the organ. Sensing a change (e.g., drop) in pressure as the needle 400 nears the outer surface of the organ or when the needle 400 punctures through the surface of the organ may provide an indication to a user or the control system 208 that triggers termination of advancement of the needle 400.

Additionally, or alternatively, a pressure sensor directly coupled to the needle 400, to another component of the endoscopic instrument system 100, or to a manual or robotically-assisted manipulator that controls operation of the endoscopic instrument system may detect a difference in pressure between an insufflated interior area within an organ or body cavity and a non-insufflated area exterior to the organ or body cavity. While the needle 400 is within the insufflated body cavity or while the distal opening 409 is within and occluded by the tissue, the pressure sensor 410 may register a pressure measurement associated with the insufflated body cavity. Once the needle 406 has penetrated through and exited the organ wall, the distal opening 409 may be outside of the insufflated body cavity and the pressure sensor 410 may register a lower pressure measurement associated with a non-insufflated body cavity. The lower pressure measurement may indicate that the needle 400 has punctured through the outer surface 411 of the body cavity. Sensing a change in pressure as the needle 410 nears the outer surface 411 of the organ or when the needle 410 punctures through the surface 411 of the organ may provide an indication to a user or the control system 208 that triggers termination of advancement of the needle 410.

In some examples it may not be necessary for the pressure sensor 410 to be near the needle 400 or the distal tip of the elongate flexible instrument 200 because the air flow in the cannulated needle may be low (e.g., a pressure difference of less than 1 atm) and a signal from a change in pressure would be simple to differentiate. In some examples a rotational coupling may both rotate the helical needle and release an air flow to apply pressure to the cannulated helical needle. In yet other examples, a micro-electromechanical system (MEMS) pressure sensor could reside on a manual or robotically-assisted manipulator that controls motion of the endoscopic instrument system 100. In this example, pressure would rise within helical needle and flow all the way back to the MEMS pressure sensor. The pressure sensor may be as far back as the pump, even if the pump is a considerable distance away from the tissue.

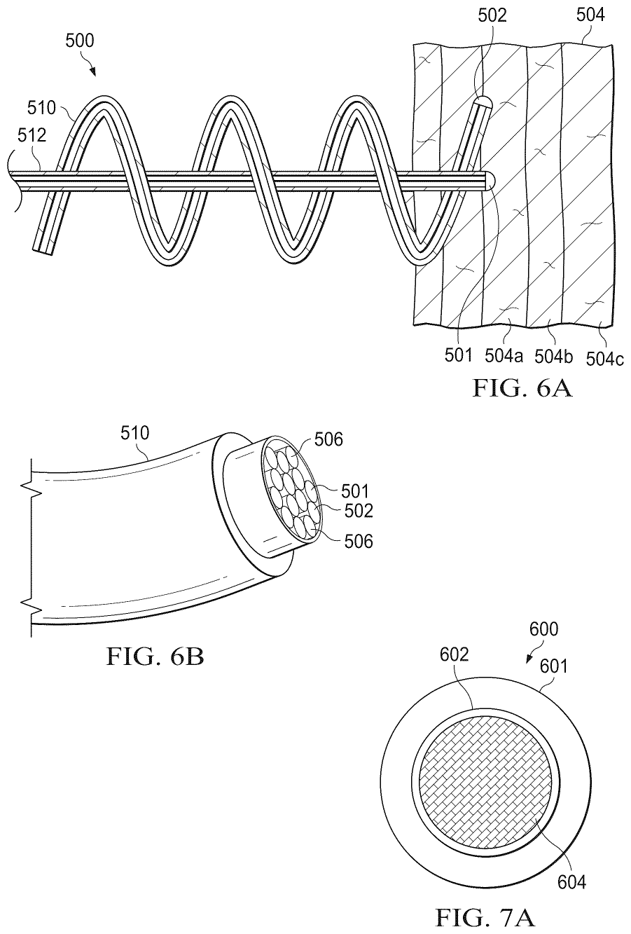

FIGS. 6A and 6B illustrate a grasping device 500 (e.g., grasping device 206, 256) that may be used together with the sensing system 210 to determine penetration information about the grasping device into tissue 504. The sensing system 210 may include an illumination device 501 and an optical sensing device 502 for detecting tissue differences. As shown in FIG. 6A, grasping device 500 may include a helical needle 510 and an elongated device 512. In some examples, the elongated device 512 may include the illumination device 501 and the needle 510 may include the optical sensing device 502. The needle 510 may extend in a wrapped configuration around the elongated device 512. The illumination device 501 may carry an illumination source at a distal end or may convey light from a remote illumination source, for example, along one or more optical fibers extending through the elongated device 512. The illumination source may be housed within the grasping device 500, attached to the outside of grasping device 500, or otherwise be in optical communication with the grasping device 500 (e.g., via optical fibers). The optical sensing device 502 may include an optical sensor located on the distal end of needle 510, or the optical sensing device 502 may convey light received at the distal end of the needle 510 (produced by the illumination source) to a remote optical sensor. The optical sensing device 502 may include one or more optical fibers extending through the device 502 to convey the light. In some examples, the illumination device 501 includes mechanical and optical couplings. In some examples, one or both of the source for the illumination device 501 and the sensor for the optical sensing device 502 may be located remotely on a manual or robotically-assisted manipulator with light signals being carried by optical fibers to the grasping device 500 as described herein.

The illumination device 501 and optical sensing device 502 may be used to detect a light parameter such as a difference in tissue color, light reflectance, or other characteristics between the layers of tissue within an organ, between different tissue types (of the same or different organs), between different organs, and/or between an organ wall and a body cavity. Different layers of tissue 504a, 504b, 504c may have different light properties. These different light properties may be measured by optical sensing device 502 and used to determine the type of tissue in which the grasping device 500 is located. For example, the four or more layers of a stomach wall (mucosa, submucosa, muscularis 1, muscularis 2, serosa) may have different light properties. Optical sensing device 502 may sense these different light properties to determine whether grasping device 500 has reached a certain layer of the target tissue or whether the grasping device 500 has penetrated through the entirety of the organ. In one example, the optical sensing device 502 may measure changes in light reflectance when grasping device 500 penetrates from inside the stomach to the abdominal cavity. The optical sensing device 502 may use illumination at known wavelengths and control algorithms to detect the needle's exit from the target tissue. The control system 208 may use the determined needle position information to stop further insertion of the grasping device 500. In some examples, sensing the type of environment that the needle is in can help reduce the risk of penetrating surrounding organs. For example, it may be desirable to avoid penetrating the gall bladder when performing full-thickness suturing of the stomach. Since the gall bladder may be blue in color, while the tissue of the stomach may be shades of pink, white, and red, an optical sensor may differentiate between the two types of tissue.

Either the illumination device, the optical sensing device 502 or both may include one or more optical fiber sensors that uses total internal reflectance to limit light loss. In small diameter fibers, the light may be well contained in the optical fiber because there may be a core glass and a cladding with a different index of refraction that creates a graded index from the core to the outside. The interface from the inner core and the outer layer may act as a mirror and the fibers are of such small diameter that reflections are happening from wall to wall as the light is transmitted down the one or more fibers.

In some examples, as shown in FIG. 6B, the helical needle 510 may host both the illumination device 501 and the optical sensing device 502 using multiple fibers 506 or multiple cores of a multi-core fiber, and the elongated device 512 may be optionally omitted. For example, light may be transmitted along one or more fibers or cores to the distal end of the needle 510 with a return/reflected signal transmitted back from the distal end of the needle 510 in one or more of the other fibers or cores. The multiple fibers or cores may be sliced at an angle to conform to the angle of needle 510. In this example, the cut fiber(s) is sharpened and polished to promote optical clarity.

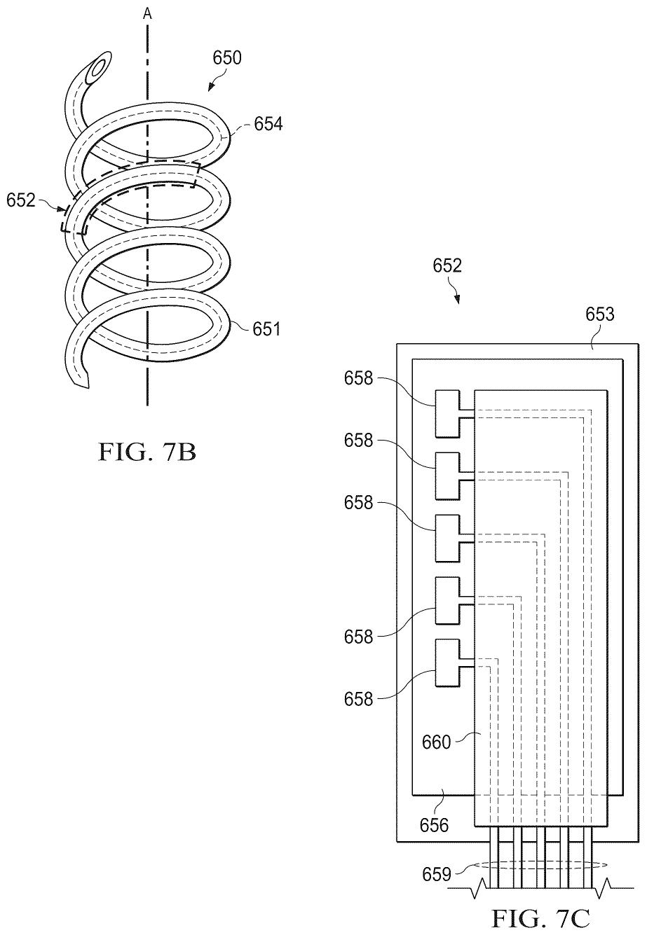

FIG. 7A illustrates a cross-sectional view of grasping device 600 (e.g., grasping device 206, 256) that may be used together with the sensing system 210 to determine penetration information about the grasping device into tissue. In this example, the sensing system 210 may include an electrical sensor 604 extending within a cannulated helical needle 601 of the grasping device 600. The electrical sensor 604 may be used to detect an electrical property such as a difference in electrical conductance, capacitance, impedance, or similar between the target tissue wall, air in a body cavity, unintended tissue, or between the layers of an organ (e.g., between the layers of the stomach to determine when the instrument has penetrated a certain layer of the stomach wall). In some examples, the electrical sensor 604 may be an insulated wire. An insulation layer 602 may be coupled concentrically around electrical sensor 604. The electrical sensor 604 may terminate at the tip of needle 601. In other examples, the wire may extend past the tip of the needle 601 to ensure contact is made between the electrical sensor 604 and the target tissue. Where a hypotube is utilized, the hypotube may be conductive. Where a hypotube is conductive, an insulation layer may be necessary. In some examples, it may be advantageous to deliver energy down the outside or inside of the wire while measuring the energy that is returning down the return path.

FIG. 7B illustrates a side view and FIG. 7C illustrates a layered surface view of a grasping device 650 (e.g., grasping device 206, 256) including a helical needle 651 that may be used together with the sensing system 210 to determine penetration information about the grasping device into tissue. In this example, a portion 652 of an electrical sensor 654 extends along a surface 653 (e.g., a stainless steel surface) of the helical needle 651 of the grasping device 650. For example, in some embodiments, the electrical sensor 654 may include one or more conductive electrodes that are positioned on an outer surface of the needle 651. The electrical sensor 654 may be used to detect an electrical property such as a difference in electrical conductance, capacitance, impedance, or similar between the target tissue wall, air in a body cavity, unintended tissue, or between the layers of an organ (e.g., between the layers of the stomach to determine when the instrument has penetrated a certain layer of the stomach wall). The electrical sensor 654 may include an insulation layer 656 on the surface 653. A series of conductive electrodes 658 may extend along the insulation layer 656, and an insulation layer 660 may extend over a portion of the electrodes 658 or the electrical traces 659 extending therefrom. In some examples, the electrodes 658 may be formed from gold or another highly conductive material.

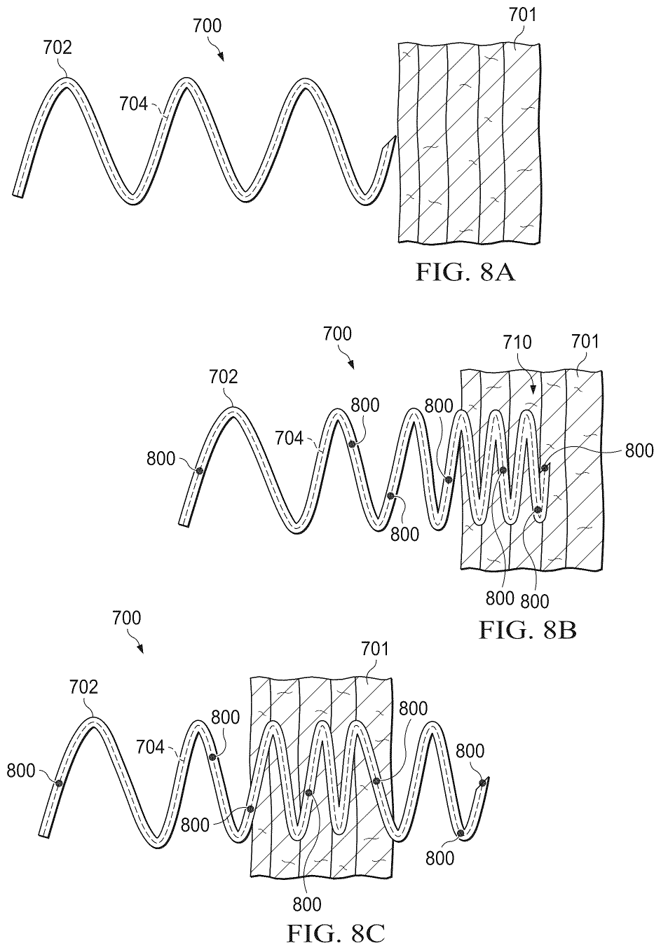

FIGS. 8A, 8B, and 8C illustrate a grasping device 700 (e.g., grasping device 206, 256) that may be used together with the sensing system 210 to determine penetration information about the grasping device 700 into tissue 701. In this example, the grasping device 700 includes a helical needle 702. The sensor system 210 may include one or more optical fiber sensors or multiple cores of a multi-core fiber 704 that extends within the helical needle. When needle 702 moves throughout organs and tissue, the needle 702 may compress or stretch like a spring, causing the optical fiber sensor 704 to become similarly deformed. This deformation of the optical fiber sensor 704 may be used to determine tissue penetration within or through a tissue wall. In some examples, the optical fiber sensor 704 may function as a strain gauge to detect a deformation or displacement of the fiber resulting from an applied force. The optical fiber sensor 704 may include fiber Bragg gratings that reflect particular wavelengths of light. As the fiber becomes compressed or stretched, the fiber Bragg gratings provide a measure of strain. The optical fiber sensor 704, acting as a strain gauge, may measure strain at many points 800 along needle 702. In some examples, the applied force may be calculated from the strain and/or deflection of the needle.

FIG. 8A illustrates needle 702 in a neutral position wherein the needle 702 is not undergoing stretching or compression. Needle 702 may be in this position as the needle 702 is navigating anatomic passageways 102 or when the needle 702 has not yet been impeded by tissue 701. In some examples, needle 702 may stay in a neutral position even while the needle 702 is being inserted into tissue 701, depending on the physical properties of needle 702 and tissue 701. The pressure measurements at points 800 obtained while needle 702 is in a neutral position may be stored and used to compare to pressure measurements obtained after needle 702 has been deflected due to contact with tissue or other impediments within the anatomy.

FIG. 8B illustrates a distal portion 710 of needle 702 in a compressed configuration. In some examples, pressure measurements at points 800 along optical fiber sensor 704 may indicate that the distal portion 710 of needle 702 is compressed. Based on the determination that the needle 702 is compressed, deflected, or otherwise deformed, the control system 208 may control the drive system 212 to stop movement, start movement, change a drive rate, continue at a same drive rate, and/or make another adjustment to the motion of the needle 702.

In FIG. 8C, some portions of needle 702 are in a compressed state while other portion(s) are in a neutral or stretched state. In some examples, when the distal portion 710 of needle 702 exits an organ 701 or enter new layers of tissue, the needle may return to a neutral state. The optical fiber sensor may sense these changes based on pressure measurement points 800. In some examples where an abrupt change in pressure is sensed, the control system and/or needle drive system may be configured to automatically stop, start, or retract needle 702. The pressure measurement points 800 illustrated in FIGS. 8A-8C are for illustrative purposes. The optical fiber sensor may measure pressure at more or fewer locations than illustrated. In some examples, the optical fiber sensor may sense pressure along the entire length of the optical fiber.

In some examples, the optical fiber sensor 704 may include an optical shape sensing fiber, which may include multiple fibers and/or multiple cores of a multi-core fiber. The shape sensing fiber may measure shape to determine the shape of the helical needle 702, which may include the diameter and/or pitch of the helical needle, as the needle changes during insertion or retraction. In some examples, the shape sensing optical fiber may measure the twist of needle 702 to determine how much torque/torsional resistance is present as needle 702 is advanced toward or into tissue. For example, the optical fiber sensor 704 may measure the distance the tip of needle 702 has extended beyond the distal end of the elongate flexible instrument 200. Any number of measurements can be used to determine the depth of needle 206 in target tissue. In another example, the distance between turns of a helical needle may be compared to determine the extent to which the needle 702 is compressed or stretched.

In some examples, the imaging system 255 (FIG. 3) may be a component of the sensor system 210 used to determine penetration of the grasping device 256 into tissue. The imaging system 255, which may be an endoscopic imaging system, may capture images that may be analyzed by an operator, the sensor system, and/or a control system to determine the extent of any penetration of the grasping device 256 into tissue. In some examples, as the helical needle is advanced into tissue, the tissue may move with the embedded tissue. From analysis of the images of the helical needle within the tissue, the layer of the tissue reached by the helical needle may be determined. In some examples, the image data may capture the number of helical revolutions that remain outside the tissue or that have entered into the tissue. The quantity of helical revolutions captured in the image data may be used to determine the depth of tissue penetration. In some examples, the image data may capture images of tissue “swirl” which may be tissue blanching caused by the profile of the embedded helical needle. The profile of the helical needle visible as a swirled protrusion of the tissue or as swirled tissue stretching or blanching caused by the helical needle may allow the viewer to assess the extent to which the helical needle has penetrated the tissue. These images of the tissue may be used to determine the depth of tissue penetration. In some examples, artificial intelligence may be used to detect pulses, changes in color, and/or changes in the shape of tissue which may be used to determine the depth of tissue penetration. In some examples, image processing may be used to analyze the 3D images to detect changes associated with the tissue swirl. For example structured light or 3D reconstruction may be performed using stereoscopic image data. These methods may be used to yield geometric data and/or a 3D model that approximates the shape of the tissue bump being created by helix engagement and tension. The 3D shape data may be analyzed (e.g., using image processing techniques) to determine the characteristic of the tissue deformation. For example, if the resulting tissue bump is tented or less defined (e.g., more “tent-like” and less “bump-like”), the likelihood that an extra-gastric organ has been captured by the helix is lower.

In some examples, the sensor system 210 may include encoders in grasper drive system 212. The encoders may count the number of rotations of needle 256 to determine a depth of insertion of the needle 256 into tissue. Additionally, or alternatively, the grasper drive system 212 may include one or more actuators to impart axial push or pull movements to the needle 256 relative to the instrument 200, and the grasper drive system 212 may be used to provide axial force data to indicate whether the needle 256 has obtained a full-thickness suture bite. Needle 256 may be tugged or pushed axially by the grasper drive system 212 to detect the force required to displace the grabbed tissue a certain distance. The system may be programmed to iteratively check and plot the force required to displace the grabbed tissue at various needle depths. The force data may indicate whether the needle 256 is too shallow, has obtained a full-thickness suture bite, or has gone too deep. In some examples, the force data may be obtained from a fiber Bragg gating (FBG) sensor. Strain information from a FBG sensor within the helix, at the tip of the helix, or proximal to the helical portion may be used to determine the force data. In some examples, the force data may help to indicate if needle 256 has been inserted into a surrounding organ. If needle 256 is inserted into a surrounding organ, the required force to pull the tissue a certain distance will be greater than if needle 206 is only inserted into the target tissue. Additionally, or alternatively, a needle 256 inserted into tissue may be pulled or pushed axially relative to the tissue by moving the distal end portion 216 of the instrument 200 via the instrument drive system 214.

The sensor system 210 is not limited to the sensor types and modalities listed. In some examples, the sensor system 210 includes multiple sensing modalities. One or more of the sensing types listed may be utilized separately or together. The sensor system 210 may perform sensing at the distal end of needle 206, along the length of needle 206, and/or at a proximal end of needle 206. In some examples, the sensors are insulated within the needle 206. In yet other examples, a straight needle may house the sensing capability while a helical shaped needle is coiled around the straight needle.

FIG. 9 is a flowchart illustrating a method 900 for grasping tissue during a medical procedure. The method 900 is illustrated as a set of operations or processes that may be performed in the same or in a different order than the order shown in FIG. 9. One or more of the illustrated processes may be omitted in some examples of the method. Additionally, one or more processes that are not expressly illustrated in FIG. 9 may be included before, after, in between, or as part of the illustrated processes. In some examples, one or more of the processes of method 900 may be implemented, at least in part, by a control system executing code stored on non-transitory, tangible, machine-readable media that when run by one or more processors (e.g., the processors of a control system) may cause the one or more processors to perform one or more of the processes.

At an optional process 902, an elongate flexible instrument may be inserted into an endoluminal passage. For example, the distal end portion 241 of the endoscopic instrument system 240 may be advanced into an anatomic opening (e.g., a patient mouth) and through the anatomic passageways 102 to perform a medical procedure, such as an endoscopic sleeve gastroplasty, at or near target tissue located in a region 108 of the anatomic structure 104. Where the medical procedure is an endoscopic sleeve gastroplasty, the target region may be one or more portions of the inner wall of the stomach. Optional process 902 may be performed with a robotically-assisted endoluminal medical system, manually by a clinician, or both.

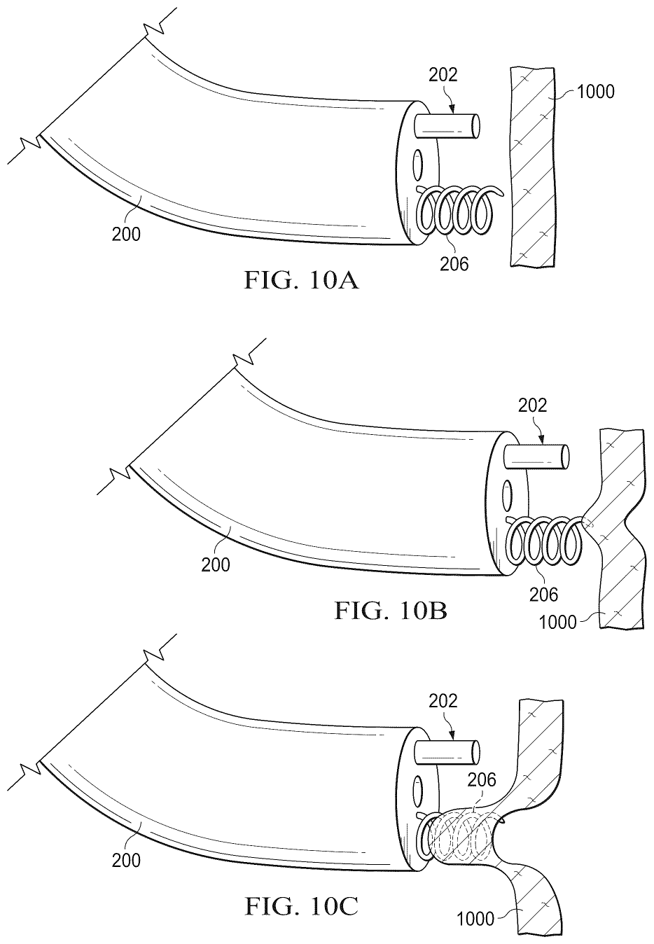

At a process 904, a tissue grasping device may extend from the elongate flexible instrument into tissue. For example, the helical needle of grasping device 206 may extend from the distal end portion 216 of endoscopic instrument system 100 into tissue. In some examples the needle can be extended longitudinally at a constant or a changing rate. In some examples, the needle may be extended longitudinally from the elongate flexible instrument 200. In some examples, extending the needle into tissue may include rotating the helical needle into the tissue while the needle advances longitudinally relative to the distal portion of the elongate flexible instrument 200. In some examples, extending the needle into tissue may include rotating the helical needle into the tissue while the needle remains longitudinally stationary relative to the distal portion of the elongate flexible instrument 200. In some examples, extending the needle into tissue may include rotating the helical needle into the tissue while the needle retracts longitudinally relative to the distal portion of the elongate flexible instrument 200. In some examples, longitudinal and/or rotational motion of the helical needle may be actuated manually and/or by grasper drive system 212. FIG. 10A, for example, illustrates the grasping device 206 extended from the elongate flexible instrument 200 and prior to engagement with the tissue wall 1000. FIG. 10B, for example, illustrates the grasping device 206 extending into the tissue wall 1000 and beginning to fold the tissue.

At a process 906, sensor information about penetration of the tissue grasping device into the tissue may be received. For example, the sensor system 210 may detect sensor information about the penetration of needle 206 into the tissue. The sensor system 210 may detect a depth of penetration of the needle 206, whether the needle 206 has penetrated all the way through a tissue wall, whether any organs outside of the target tissue have been impacted, and/or what layer of tissue has been reached. The sensor system may detect penetration using, for example, a pressure sensor system, an optical sensor system, an electrical sensor system, an optical fiber sensor system, an imaging system, encoders in the drive system, differential measurement, or combinations of sensor information from any sensor systems as described above. In some examples, as shown in FIG. 10C, the helical needle 206 pulls the tissue 1000 into a fold as the needle extends into the tissue. As the helical needle 206 penetrates the tissue 1000, the sensor system may detect sensor information about the penetration of the needle using any of the sensor systems previously described. For example, the sensor system 210 may include a strain sensor, such as an optical fiber strain sensor extending within the needle 206. As the needle 206 pulls or pushes tissue 1000, resistance may be measured by the optical fiber strain sensor. The strain measurements obtained by the optical fiber strain sensor may be used to determine whether a full thickness penetration has been achieved.

At a process 908, the penetration information may be used to control the tissue grasper drive system. For example, the control system 208 may control the grasper drive system 212 based on the sensor information received. In some examples the control system 208 may control whether the penetration is initiated, terminated, or reversed. In some examples, the control system 208 may control the speed and depth of penetration based on the received sensor information. The control system may control both depth and penetration of the needle simultaneously through needle drive system 212. In other examples, the control system 208 may provide user feedback regarding needle penetration depth, errors in tissue grabbing/handling, and whether resistance caused by tissue is such that the instrument is safe to be retracted to fold tissue.

At an optional process 910, a medical procedure may be performed with an instrument system while the tissue is grasped by the tissue grasping device. For example, while the needle 206 grasps the tissue 1000 as shown in FIG. 10C, a portion of the instrument system 202 (e.g., the suturing needle 253 from the instrument system 252) may sweep through the folded tissue 1000 to tie a suture across the fold. In other examples, instrument systems may ablate the tissue, biopsy the tissue, treat the tissue, or otherwise perform a medical procedure while the tissue is grasped by the needle 206.

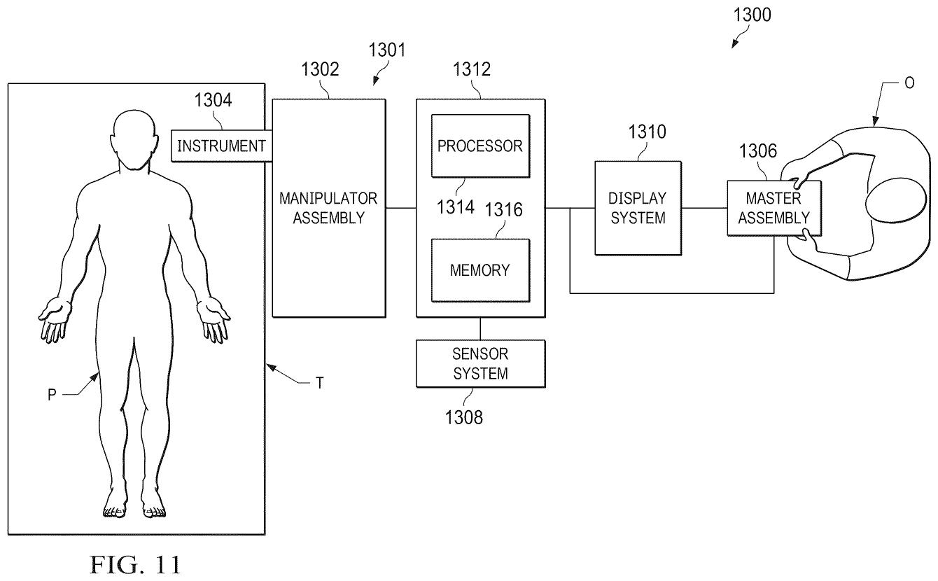

FIG. 11 illustrates a robotically-assisted medical system, according to some examples. In some examples, the systems and methods disclosed herein may be used in a medical procedure performed with a robotically-assisted medical system as described in further detail below. As shown in FIG. 13, a robotically-assisted medical system 1300 may include a manipulator assembly 1302 for operating a medical instrument 1304 (e.g., endoscopic instrument system 100 or any of the instruments described herein) in performing various procedures on a patient P positioned on a table T in a surgical environment 1301. The manipulator assembly 1302 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 1306, which may be inside or outside of the surgical environment 1301, generally includes one or more control devices for controlling manipulator assembly 1302. Manipulator assembly 1302 supports medical instrument 1304 and may optionally include a plurality of actuators or motors that drive inputs on medical instrument 1304 in response to commands from a control system 1312. The actuators may optionally include drive systems that when coupled to medical instrument 1304 may advance medical instrument 1304 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 1302 may support various other systems for irrigation, treatment, or other purposes. Such systems may include fluid systems (including, for example, reservoirs, heating/cooling elements, pumps, and valves), generators, lasers, interrogators, and ablation components.

Robotically-assisted medical system 1300 also includes a display system 1310 for displaying an image or representation of the surgical site and medical instrument 1304 generated by a sensor system 1308 which may include an endoscopic imaging system. Display system 1310 and master assembly 1306 may be oriented so an operator O can control medical instrument 1304 and master assembly 1306 with the perception of telepresence. Any of the previously described graphical user interfaces may be displayable on a display system 1310 and/or a display system of an independent planning workstation.

The sensor system 1308 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 1304. The sensor system 1308 may also include temperature, pressure, force, or contact sensors or the like.

Robotically-assisted medical system 1300 may also include control system 1312. Control system 1312 includes at least one memory 1316 and at least one computer processor 1314 for effecting control between medical instrument 1304, master assembly 1306, sensor system 1308, and display system 1310. Control system 1312 also includes programmed instructions (e.g., a non-transitory machine-readable medium storing the instructions) to implement a plurality of operating modes of the robotically-assisted medical system including a navigation planning mode, a navigation mode, and/or a procedure mode. Control system 1312 also includes programmed instructions (e.g., a non-transitory machine-readable medium storing the instructions) to implement some or all of the processes described in accordance with aspects disclosed herein, including, for example, expanding the expandable device, regulating the temperature of the heating system, regulating valves to control fluid delivery, controlling fluid flow rate, controlling insertion and retraction of the treatment instrument, controlling actuation of a distal end of the treatment instrument, receiving sensor information, altering signals based on the sensor information, selecting a treatment location, and/or determining a size to which the expandable device may be expanded.

Control system 1312 may optionally further include a virtual visualization system to provide navigation assistance to operator O when controlling medical instrument 1304 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 1312 may use a pre-operative image to locate the target tissue (using vision imaging techniques and/or by receiving user input) and create a pre-operative plan, including an optimal first location for performing bronchial passageway and vasculature occlusion. The pre-operative plan may include, for example, a planned size to expand the expandable device, a treatment duration, a treatment temperature, and/or multiple deployment locations.

In the description, specific details have been set forth describing some examples. Numerous specific details are set forth to provide a thorough understanding of the examples. It will be apparent, however, to one skilled in the art that some examples may be practiced without some or all these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in examples of this disclosure may be implemented in software to execute on a processor of a computer system such as control processing system. When implemented in software, the elements of the examples of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative examples of the invention have been described and shown in the accompanying drawings, it is to be understood that such examples are merely illustrative of and not restrictive on the broad invention, and that the examples of the invention are not limited to the specific constructions and arrangements shown and described, since various other modifications may occur to those ordinarily skilled in the art.

1. A system comprising:
an elongate flexible instrument;
a tissue grasping device including a helical needle extending from a distal end of the elongate flexible instrument;
a grasper drive system coupled to the tissue grasping device to drive motion of the tissue grasping device;
a sensor system coupled to the tissue grasping device; and
a control system configured to receive sensor information from the sensor system and control the grasper drive system in response to the received sensor information.

2. The system of claim 1, wherein controlling the grasper drive system includes providing an actuation signal to the grasper drive system.

3. The system of claim 1, wherein controlling the grasper drive system includes adjusting a setting of the grasper drive system.

4. (canceled)

5. The system of claim 1, further comprising an instrument system, the instrument system in communication with the control system.

6. The system of claim 5, wherein the instrument system is at least one of
a suturing system,
a biopsy system,
a treatment system, or
an ablation system.

7. The system of claim 5, wherein the instrument system is configured to initiate a procedure based on the received sensor information.

8. The system of claim 1, wherein the elongate flexible instrument includes an endoscopic imaging system.

9. The system of claim 1, wherein the tissue grasping device is cannulated, and a component of the sensor system extends within the tissue grasping device.

10. The system of claim 1, wherein the helical needle is a first helical needle and the tissue grasping device includes a second helical needle.

11. The system of claim 1, wherein the tissue grasping device comprises a straight needle and the helical needle is wrapped around an axis of the straight needle.

12. The system of claim 1, wherein the sensor system includes a pressure sensor configured to detect a change in fluid pressure in a lumen extending through the tissue grasping device as the tissue grasping device penetrates tissue.

13. (canceled)

14. The system of claim 1, wherein the sensor system includes an optical sensor configured to detect a change in a parameter of reflected light as the tissue grasping device penetrates tissue.

15. (canceled)

16. The system of claim 1, wherein the sensor system includes an electrical sensor.

17. The system of claim 16, wherein the electrical sensor is configured to detect a change in an electrical property as the tissue grasping device penetrates tissue.

18. The system of claim 1, wherein the sensor system includes an optical fiber sensor.

19. The system of claim 18, wherein the optical fiber sensor is configured to detect a change in strain as the tissue grasping device penetrates tissue.

20. The system of claim 18, wherein the optical fiber sensor is configured to detect a change in shape of the tissue grasping device as the tissue grasping device penetrates tissue.

21. (canceled)

22. The system of claim 1, wherein the sensor system includes encoders in the grasper drive system.

23. The system of claim 1, wherein the tissue grasping device includes a slidable elongated core.

24. The system of claim 1, wherein a slidable sheath extends around the tissue grasping device.

25-31. (canceled)
